(19) United States
(12) Patent Application Publication
(10) Pub. No.: US 2008/0193572 A1
(43) Pub. Date: Aug. 14, 2008

(54) Extracts of Ginkgo Biloba

(76) Inventors: Christophe Carite, Rilhac-Rancon (FR); Santiago Rull Prous, Barcelona (ES); Smail Alaoui Ismaili, Cerdanyola del Valles (ES); Pedro Forner, Barcelona (ES); Christine Gartner, Dusseldorf (DE); Sybille Buchwald-Werner, Dusseldorf (DE)

Correspondence Address: COGNIS CORPORATION;PATENT DEPARTMENT, 300 BROOKSIDE AVENUE, AMBLER, PA, 19002, US

(21) Appl. No.: 11/995,589

(22) PCT Filed: Jul. 1, 2006
(86) PCT No.: PCT/EP2006/006429

§ 371 Date: Jan. 14, 2008

(30) Foreign Application Priority Data
Jul. 12, 2005 (FR) 0552158

(51) Int. Cl. A61K 36/16; A61P 27/02

(52) U.S. Cl. 424/752

(57) ABSTRACT

Novel extracts from the leaves of Ginkgo biloba, comprising 20 to 30%, by weight, of flavone glycosides; 2.5 to 4.5%, by weight in the aggregate, of ginkgolides A, B, C and J; 2.0 to 4.0%, by weight, of bilobalides; less than 10 ppm of alkyl phenol compounds; and more than 10%, by weight, of oligomeric proanthocyanidins, useful for pharmaceutical, dietary supplements and/or functional food/nutraceutical compositions, and a novel process for obtaining such extracts.

### CROSS-REFERENCE TO RELATED APPLICATIONS

This application is filed under 35 U.S.C. §371, claiming priority from PCT/EP2006/006429 filed Jul. 1, 2006, which claims priority from FR 0552158 filed Jul. 12, 2005, the entire contents of each application are incorporated herein by reference.

### OBJECT OF THE INVENTION

The present invention relates to new extracts of the leaves of Ginkgo biloba, a process for obtaining said extracts and their use for making oral preparations for pharmaceuticals dietary supplements and/or functional foods/nutraceuticals, including foods for particular nutritional and/or medical purposes, and the like.

### STATE OF THE ART

The ginkgo tree, which has survived in the temple gardens of China and Japan, is a phenomenon. Darwin called it “a living fossil”, since all of its properties are associated with longevity.

Although Ginkgo biloba is the only extant species of ginkgos today, many ginkgo relatives have been found in the fossil record. The Ginkgoales are a group of gymnosperms that date back to the Permian and are thought to be more closely related to the conifers than any other gymnosperms are. The modern-day Ginkgo biloba can grow up to 30 meters and can live for a millennium. The leaves are used as a herbal medicine and the seeds were also used for that purpose in the past. In China so-called “Bai-guo-ye” is used to treat respiratory problems, hearing loss, coughing, tuberculosis, poor circulation, memory loss, gonorrhoea, stomach pain, skin diseases, leukorrhoe, angina pectoris, dysentery, high blood pressure, anxiety and other conditions. The powdered leaves are inhaled for asthma, and ear, nose and throat disorders.

In Western medicine the leaves became an object of research in the late 1950s. Willmar Schwabe analysed the constituents and activity of the natural substances of the leaves and started to commercialize the ginkgo extract. Under the brand Tebonin® tincture and tablets have been offered at a concentration of 10:1 (raw material:extract ratio). Later other companies also developed the extract with current concentration being mostly 50:1 (raw material:extract ratio). Meanwhile many controlled studies and research on the chemistry, pharmacology and clinical effects of the leaves have been conducted, mostly using the extract EGb761, also called Kaveri, Tebonin®, Tanakan®, Rökan or Ginkgold. In 1988 Elias Corey of Harvard University won the Nobel prize for synthesising ginkgolide B, which is being investigated for its use, inter alia, to prevent rejection of transplanted organs as well as for asthma and toxic shock.

The main active ingredients of Ginkgo biloba extracts are flavonoids (such as quercetin, kaempferol, isorhamnetin, myricetin) and their glycosides, terpeniods (such as ginkgolides A, B, C, J, and M and bilobalides), and some small phenolic compounds.

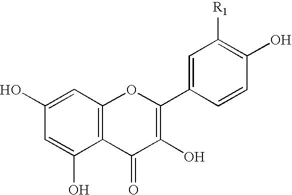

### Quercetin R₁=OH; Kaempferol R₁=H; Isorhamnetin R₁=OCH₃

Numerous documents discuss extracts of Ginkgo biloba and processes for obtaining them. Of particular interest are EP 0431535 B1 and EP 0431536 B1 (both Schwabe) which are directed to extracts of the leaves of Ginkgo biloba comprising (a) 20 to 30% by weight of flavone glycosides, (b) 2.5 to 4.5% by weight in the aggregate, of ginkgolides A, B, C and J, (c) 2.0 to 4.0% by weight, bilobalide, (d) less than 10 ppm of alkyl phenol compounds and (e) less fain 10% by weight of condensed tannins, more preferably (oligomeric) proanthocyanidins (OPCs), and a method for obtaining them. In fact the composition claimed by Schwabe has become a standard for all pharmaceutical applications of gingko extracts. Particular attention is directed to the amounts of components (d) and (e): as, while the gingkolic acids are suspected of causing irritations, proanthocyanidins (OPCs) are responsible for haemaglutinating and serum-precipitating properties if the Ginkgo extract is administered intravenously or intramuscularily, circumventing the oral route. The negative properties of OPCs, however, are also reported in EP 0477968 B1 (Schwabe) which discusses entirely removing these compounds from the extracts by a special process.

EP 0360556 B1 (Indena SpA) discloses in Example 1 a ginkgo composition comprising 24% by weight, of flavone glycoside, 3.6% by weight, of ginkgolides, 3.1% by weight, of bilobalides and a so-called “procyanidolic index”, which is considered to be equivalent to the OPC content, of 9% by weight. The documents EP 1037646 B1 and EP 1089748 B1 Roth Schwabe) disclose Ginkgo compositions which are characterised by a reduced content of other components, like 4′O-methyl-pyridoxines, biflavones and terpene lactones.

Although the ginkgo extracts presently on the market fulfilled the needs with respect to the known health properties of ginkgo, today's consumers continue to demand products with improved and/or additional properties. In case of ginkgo for example it is a desirable to develop new extracts which in addition to providing the known health benefits, protect the person taking an extract orally against the various negative effects of free radicals. In addition to that objective, a second objection of this invention was to develop ginkgo extracts which improve the overall status of the human body, e.g. with respect to the microcirculation of the blood. Such a product could be easily obtained by adding specific actives, which for example are well known for contributing radical scavenging and blood circulation stimulating properties to the existing extracts, but such products would be much more expensive due the increased technical effort to produce them. In addition, such extracts would no longer represent true ginkgo extracts covered by the standard pharmaceutical specification. Therefore, still an additional objective underlying the present invention was to provide a ginkgo extract having the additional properties listed above without adding cost-increasing active ingredients.

### BRIEF SUMMARY OF THE INVENTION

The present invention is directed to novel extracts from the leaves of Ginkgo biloba comprising flavone glycosides, ginkgolides, bilobalides, and alkyl phenol compounds, each in amounts according to the accepted standards for Ginkgo biloba extracts, up to 5% of water, and more than 10%, preferably 11-20%, and more preferably 12-18%, by weight, of oligomeric proanthocyanidins (OPCs), as well as a method for makling them comprising a novel eight-step process, comprising beginning with ginko leaves or dry extracts of Ginkgo biloba, conducting a series of extractions, with appropriate solvents and necessary pH adjustments, concentrations, dilutions, drying, separations, precipitation, washing, and final addition of constituents in order to obtain an extract particularly rich in OPCs.

### DETAILED DESCRIPTION OF THE INVENTION

All quantities herein, except those reflecting actual quantities employed in practiced Examples, should be read as preceded by “about.” The present invention claims new extracts from the leaves of Ginkgo biloba, comprising
(a) 20 to 30% by weight of flavone glycosides,
(b) 2.5 to 4.5% by weight in the aggregate of ginkgolides A, B, C and J,
(c) 2.0 to 4. 0% by weight of bilobalides,
(d) less than 10 ppm of alkyl phenol compounds and
(e) more than 10% by weight oligomeric proanthocyanidins (OPCs), and a process for preparing such extracts from the leaves or dry extracts of Ginkgo biloba.

As the result of various experiments and tests it has surprisingly been found that these extracts from ginkgo leaves fully satisfied the expected improved health benefits, by increasing the content of oligomeric proanthocyanidins above the previously-believed limit of 10%, to preferably 11-20% and more preferably to 12-18% by weight. This invention overcomes a prejudice from the state of the art, which—due to generally accepted scientific knowledge, as reflected in the Schwabe and Indena SpA references, op cit—has been to reduce the amount of said OPCs to a content of less than 10% by weight, or even to remove these compounds entirely. Specifically, the improved health benefits of the ginkgo extracts with increased OPC content, as described in this invention, refer to improved antioxidant effects of the extracts, resulting in improved anti-inflammatory activity and improved beneficial effects for vascular tissues, including reduced capillary fragility and better connective tissue stabilization. These are of particular relevance to eye health, by improving retinal microcirculation, accelerated resynthesis of rhodopsin, modulation of retinal enzyme activity, and other benefits. These s improved benefits of ginkgo extracts with increased OPC content confer benefits including but not limited to improved night vision and dark adaptation, as well as improved renal blood flow which is relevant in diabetic retinopathy, other types of retinopathies, age-related macular degeneration and glaucoma

Oligomeric Proanthocyanidins (OPCs)

Oligomeric proanthocyanidins, also known as procyanidins, leucoanthocyanins or condensed tannins, are oligmers or polymers with flavan-3-ols such as (+)-catechin or (−)-epicatechin forming the basic units. Their name reflects the fact that they are converted to the coloured anthocyanidins upon acid hydrolysis. Usually the linkage between successive monomers is via C₄ to C₈, but may also occur via C₄ to C₆. The structures may be represented by the following.

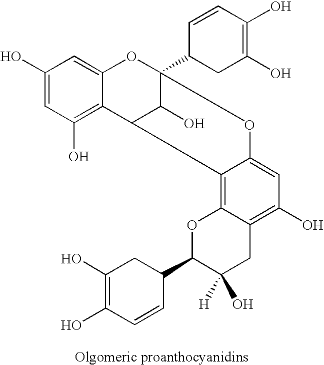

The analysis of the OPC content of the ginkgo extracts according to the present invention has been carried out according to the instructions as set out in EP 0360556 B1 (Indena SpA), which is explicitly incorporated herein by reference.

Extraction Process

Another aspect of the invention is a process for making extracts from the leaves of Ginkgo biloba, which extracts comprise:
(a) 20 to 30% by weight of flavone glycosides,
(b) 2.5 to 4.5% by weight in the aggregate of gnkgolides A, B, C and J,
(c) 2.0 to 4. 0% by weight of bilobalides,
(d) less than 10 ppm of alkyl phenol compounds and
(e) more than 10% by weight of oligomeric proanthocyanidins (OPCs).

Such inventive process consists of the following steps:
(i) eider leaves or dry extracts of Ginkgo biloba are subjected to extraction with an aqueous polar solvent in order to give a first liquid intermediate (LI-1);
(ii) said intermediate LI-1 is separated from the polar solvent and subjected to a liquid-liquid ex on with a non-polar C₄-C₁₀ hydrocarbon in order to obtain a second (aqueous) liquid intermediate (LI-2);
(iii) said intermediate LI-2 is adjusted to a pH of 2.5 to 6.0 and next subjected to a liquid-liquid extraction with a polar C₂-C₆ aliphatic alcohol in order to obtain an (aqueous) liquid intermediate (LI-3) rich in OPCs and an (organic) liquid intermediate (LI-4) rich in glycosides;
(iv) said intermediate LI-4 is concentrated, diluted with water and mixed with a non-polar C₄-C₁₀ hydrocarbon in order to obtain an (organic) liquid intermediate (LI-5) and an (aqueous) liquid intermediate (LI-6), while LI-5 can be dried, if necessary, in order to adjust the final terpene lactone content,
(v) said liquid intermediate LI-6 is dried to give a first solid intermediate (SI-1);
(vi) said liquid intermediate LI-3 is separated from the polar solvent, diluted with water, adjusted to a pH value of 6 to 8 and cooled to a temperature of at most 10° C. for a period sufficient to precipitate the OPCs from the solution;
(vii) said precipitated OPCs are filtered off, washed and dried in order to give a second solid intermediate (SI-2); and finally
(viii) the second solid intermediate SI-2 is added to the first solid intermediate SI-1 in such amount that the final product contains more than 10% by weight of OPCs.

More particularly the extracts obtained according to the invention typically show a content of OPCs of 11 to 20, more preferably 12 to l8% and more preferably 13 to 15% by weight of OPCs, usually comprising
(i) less than 50 ppm 4′O-methyl-pyroxidines,
(ii) less than 100 ppm biflavones, and
(iii) 5 to 10% by weight of tee lactones.

The water content of the exacts is typically at most 5% by weight.

A particular advantage of the new process is that one can start either from ginkgo leaves (typically showing a content of flavone glycosides, ginkgolides and bilobalides of at least 10% by weight) or commercially available dry ginkgo extracts (typically showing a content of flavone glycosides, ginkgolides and bilobalides of 5 to 20% by weight in order to end up with a final product which matches the specifications, in particular showing an OPC content of more than 10, preferably about 12-18% by weight.

In preferred embodiments of the present invention, the polar solvent of step (i) is acetone or ethanol. It has been found that acetone is very suitable for the extraction of the leaves, while ethanol is the preferred solvent for the extraction of the dry intermediates one can buy on the market. The non-polar hydrocarbon of steps (ii) and (iv) is preferably n-heptane, which is useful to ensure that all unwanted ginkgolic acids are removed and concentrated in an organic waste phase. Moreover, said polar alcohol of step (iii) is preferably n-butanol. The major improvement of the new process over the prior one is to separate a fraction rich in OPCs from the main stream, to concentrate, purify and isolate said OPCs, and finally add them back to the main stream, in order to increase the OPC content from typically 4 to 8% by weight to more than 10%, and typically about 12-18% by weight.

Encapsulation

Dried mixtures according to the present invention may also be formulated as powders, granules or semisolids for incorporation into capsules. When used in the form of powders, the compositions may be formulated together with any one or more excipients, or they may be presented in an undiluted form. For presentation in the form of a semisolid, the dried mixtures can be dissolved or suspended in a viscous liquid or semisolid vehicle, such as a polyethylene glycol, or a liquid carrier, such as a glycol, e.g., propylene glycol, or glycerol, or a vegetable or fish oil, for example, an oil selected from olive oil, sunflower oil, safflower oil, soy oil and other oils. Such extracts can be macro-encapsulated, i.e., filled into capsules of either the hard gelatin or soft gelatin type or made from hard or soft gelatin equivalents gelatin-free), soft gelatin or gelatin-equivalent capsules preferred for viscous liquid or semisolid fillings.

In a special embodiment of the present invention said active compositions are micro-encapsulated into spherical aggregates with a diameter from about 0.1 to about 5 mm which contain at least one solid or liquid core surrounded by at least one continuous membrane. More precisely, they are finely dispersed liquid or solid phases coated wit film-forming polymers, in the production of which the polymers are deposited onto the material to be encapsulated after emulsification and coacervation or interfacial polymerization. In another embodiment, liquid active principles are absorbed in a matrix (“microsponge”) and, as microparticles, may be additionally coated with film-forming polymers. The microscopically small capsules, also known as nanocapsules, can be dried in the same way as powders. Besides single-core microcapsules, there are also multiple-core compositions, also known as microspheres, which contain two or more cores distributed in the continuous membrane material. In addition, single-core or multiple-core microcapsules may be surrounded by an additional second, third, or more membranes. The membrane surrounding the core(s) may consist of natural, semisynthetic or synthetic materials. Natural membrane materials include, for example, gum arabic, agar agar, agarose, maltodextrins, alginic acid and salts thereof, for example, sodium or calcium alginate, fats and fatty acids, cetyl alcohol, collagen, chitosan, lecithins, gelatin, albumin, shellac, polysaccharides, such as starch or dextran polypeptides, protein hydrolyzates, sucrose and waxes. Semisynthetic membrane materials include, inter alia, chemically-modified celluloses, more particularly cellulose esters and ethers, for example, cellulose acetate, ethyl cellulose, hydroxypropyl cellulose, hydroxypropyl methyl cellulose and carboxymethyl cellulose, and starch derivatives, more particularly starch ethers and esters. Synthetic membrane materials include, for example, polymers, such as polyacrylates, polyamides, polyvinyl alcohol or polyvinyl pyrrolidone.

Examples of known microcapsules include the commercial products (with the membrane material(s) shown in brackets): Hallcrest Microcapsules (gelatin, gum arabic), Coletica Thalspheres (maritime collagen), Lipotec Millicapseln (alginic acid, agar agar), Induchem Unispheres (lactose, microcrystalline cellulose, hydroxypropylmethyl cellulose), Unicerin C30 (lactose, microcrystalline cellulose, hydroxypropylmethyl cellulose), Kobo Glycospheres (modified starch, fatty acid esters, phospholipids), Softspheres (modified agar agar), Kuhs Probiol Nanospheres phospholipids) and Primaspheres or Primasponges (chitosan, anionic polymers).

The encapsulation of the compositions according to the present invention is preferred where the actives are administered orally and are to be liberated at a special part of the intestines. Therefore, a person skilled in the art can easily select the adequate encapsulation system by comparing the stability of the capsules under the pH-conditions of the respective part of the intestines. Suitable processes for selecting the appropriate encapsulation system and for encapsulation are disclosed for example in WO 01/01926, WO 01/01927, WO 01/01928, WO 01/01929 (Primacare) or EP 1064088 B1 (Max Planck Gesellschaft), which are incorporated herein by reference.

### Commercial Application

As outlined above, the new extracts combine the known advantageous properties of no extracts found the market with new surprising features, particularly for improving the overall status of the human body, especially with respect to protection against free radicals and improved retinal microcirculation.

Therefore, a further focus of the invention is the use of the new extracts rich in OPCs for making pharmaceutical preparations and/or dietary supplements and/or foods (incl. functional food/nutraceuticals including foods for particular nutritional and/or medical purposes, and the like), in which they may be present in amounts from 10 to 1,000 mg, preferably 30 to 500 mg and more preferably 60 to 240 mg (calculated on the final composition). The extracts are administered to the body either by topical application or oral administration.

Still another object of the invention is use of said extracts for making a medicament for the improvement of retinal microcirculation and the status of the human body.

The following exemplify aspects of the instant invention, without in any way limiting it.

### EXAMPLES

### Preparation Example A1

### Preparation of Ginkgo Extracts with Increased OPC Content from Ginkgo Leaves

Step I. 1000 g of leaves of Ginkgo biloba having a content of flavone glucosides, ginkgolides and bilobalides of in total 0.8% by weight were placed in a stirred vessel and extracted at 50° C. for 2 hours using 5 liters of aqueous acetone (60% w/w). The liquid phase was separated from the residue and subjected to filtration and solvent evaporation in order to give a liquid intermediate (LI-1) having a dry residue of about 30% by weight Subsequently, said LI-1 was extracted with n-heptane in order to obtain an organic phase comprising all unwanted ginkgolic acids and a second (aqueous) liquid intermediate phase (LI-2) containing the flavone glycosides, ginkgolides A, B, C and J, the bilobalides and the oligomeric proanthocyanidins.

Step II. The intermediate LI-2 thus obtained, after adjusting the pH to 2.5-6, was subjected three times to an extraction with n-butanol in order to obtain a third (aqueous) liquid intermediate phase (LI-3), rich in OPCs and a fourth (organic) liquid intermediate (LI-4), the latter being washed several times with water in order to remove the unwanted by-products and concentrated in order to obtain a concentrated fraction with a dry residue of about 20% by weight. The concentrate was subsequently diluted with water to a dry residue of about 10% by weight and mixed with n-heptane (70:30 w/w). After separation a fifth (organic) liquid intermediate (LI-5) rich in ginkgolides and bilobalides and a sixth (aqueous) liquid intermediate (LI-6) rich in the flavone glycosides ginkgolides A, B, C and J, bilobalides and oligomeric proanthocyanidins was obtained. Finally, fraction LI-6 was concentrated and dried with the final solid having an OPC content of about 7% by weight.

Step III. The liquid intermediate LI-3, which has been obtained in Step II, was liberated from all traces of organic solvents, diluted with water to obtain a dry residue of about 30% by weight and adjusted to a pH of about 6.8 to 7.2 by adding aqueous sodium hydroxide solution. Subsequently, the liquid fraction was cooled overnight to 8° C. The next day a precipitate mainly consisting of OPCs was filtered off, washed, and dried and added to the solids obtained as the final product of Step II. The combined products had the following composition (with the average of three samples in brackets):
Ginkgoflavonglycosides: 22 to 27 (24) % by weight.
Bilobalides: 2.6-3.2 (2.9) % by weight
Ginkgolides: 2.8-3.4 (3.0) % by weight
OPCs: 12-13 (12.2) % by weight
Ginkgolic acids: <10 ppm

### Preparation Example A2

### Preparation of Ginkgo Extracts with Increased OPC Content from Dry Ginkgo Extracts

1000 g of a commercially available dry extract of Ginkgo biloba, having a yellow to brown appearance and comprising less t 4.5% by weight of flavone glycosides, was placed in a stirred vessel and extracted with aqueous ethanol (80% w/w). The liquid fraction thus obtained was filtered and the solvent removed. The intermediate thus obtained was diluted with water to a dry residue of about 10% by weight and afterwards extracted with n-heptane to eliminate the ginkgolic acids. Subsequently, the aqueous phase thus obtained was treated as explained in Steps II and m of Example A1.

### Application Examples

### Demonstration of the Antioxidant Properties

Reactive oxygen species (ROS) and reactive nitrogen species (RNS) are reactive compounds which may damage important biomolecules such as proteins, lipids, carbohydrates and DNA, if not counteracted by antioxidants. Some but not all ROS and RNS are free radicals, i.e., atoms or molecules containing one or more unpaired electrons. Formation of ROS and RNS occurs as an integral part of human metabolism, for example by the mitochondrial respiratory chain, during the oxidative burst of activated phagocytes as part of the normal functioning of the immune system, or by enzymes such as xanthine oxidase. Exogenous factors such as sun light, cigarette smoke, or certain environmental pollutants may contribute to the human body's exposure to ROS and RNS. ROS/RNS are counteracted by a plethora of antioxidants, and oxidative stress occurs only when this balance shifts in favour of ROS/RNS. Then, damage to vital biomolecules and biological systems may be induced, and such damage, when accumulating over long periods of time, has been implicated in the development of many degenerative diseases as well as in the process of ageing itself.

The antioxidative properties of active substances such as the gingko extract of this invention may be measured by various tests, either in vitro or in cell culture systems or elsewhere. Each test is usually specific for a certain type of ROS and/or RNS. Because the human body is exposed to the whole spectrum of these reactive substances—also referred to as “pro-oxidants”—it may be desirable for an antioxidant to be effective against a variety of pro-oxidants. Therefore, in order to evaluate the properties of the products, the gingko extract can be subjected to a variety of tests, measuring its ability to reduce radical cations DPPH Test), its ability to scavenge hydroxyl radicals (HO.), superoxide (O₂.), hydrogen peroxide (H₂O₂), as well as its ability to quench singlet oxygen. In addition, the metal chelating properties can be assessed.

### I.

### DPPH Test

The DPPH test measures the ability of a test substance to scavenge free radicals, specifically to reduce cation radicals. The test uses DPPH (2,2-diphenyl-1-picrylhydrazyl), a stable radical which appears ‘violet’ due to its absorption maximum at 515 nm, and which is transformed into a colorless compound upon reduction by the antioxidant. Thus, the antioxidant activity of the test substance is followed by the decrease of absorbance at 515 nm. The test results are given in Table I below.

TABLE I
Radical scavenging activity of ginkgo extracts with respect to
variations in OPC content

| | | | | | |
|---|---|---|---|---|---|
| Control* | C1* | C2* | 1* | 2* | 3* |

Gingko extract composition (in % b.w.)

| | | | | | | |
|---|---|---|---|---|---|---|
| Flavone | — | 25.0 | 25.0 | 25.0 | 25.0 | 25.0 |
| glucosides | | | | | | |
| Ginkgolides | — | 3.0 | 3.0 | 3.0 | 3.0 | 3.0 |
| Bilobalides | — | 3.0 | 3.0 | 3.0 | 3.0 | 3.0 |
| Alkyl | — | <5 ppm | <5 ppm | <5 ppm | <5 ppm | <5 ppm |
| phenols | | | | | | |
| OPCs | — | 9 | 10 | 12 | 15 | 18 |

Test Results (in % inhibition compared to control)
Concentration of gingko extract in the test solution [% w/v]

| | | | | | | |
|---|---|---|---|---|---|---|
| 0.0003 | 0 | 18 | 20 | 29 | 35 | 38 |
| 0.001 | 0 | 55 | 57 | 71 | 75 | 77 |
| 0.01 | 0 | 78 | 79 | 85 | 88 | 91 |

*Control: no extract in test; C1, C2: Control gingko extracts 1 and 2, not compliant with the present invention (OPC content ≦10%); 1, 2, 3: ginkgo extracts 1, 2 and 3, compliant with the present invention (OPC content >10%)

### II.

### Hydroxyl Radical Scavenging Activity

The ability to scavenge hydroxyl radicals (HO.) can be assessed in vitro by the so-called ‘deoxyribose assay’. HO. may be considered the most reactive of all ROS/RNS, so that it can attack almost all cellular compound, including DNA constituents such as deoxyribose. In the test HO. is generated by a mixture of ascorbic acid, H₂O₂, and Fe³⁺—EDTA, i.e., via the Fenton reaction (H₂O₂ in the presence of iron). HO. attacks deoxyribose, degrading it into fragments that yield a pink chromogen upon heating with thiobarbituric acid (TBA) at a low pH. Added hydroxyl radical “scavengers” compete with deoxyribose for the hydroxyl radicals produced and diminish chromogen formation. The tests are performed both in the presence and absence of EDTA to test for the ability of OPCs to chelate (bind) transition metal ions such as iron. The results are given in Table II below and represent the average of two tests.

TABLE II
Hydroxyl radical scavenging and metal chelating activity of ginkgo
extracts with respect to variation in OPC content

| | | | | | |
|---|---|---|---|---|---|
| Control* | C1* | C2* | 1* | 2* | 3* |

Gingko extract composition (in % b.w.)

| | | | | | | |
|---|---|---|---|---|---|---|
| Flavone | — | 25.0 | 25.0 | 25.0 | 25.0 | 25.0 |
| glucosides | | | | | | |
| Ginkgolides | — | 3.0 | 3.0 | 3.0 | 3.0 | 3.0 |
| Bilobalides | — | 3.0 | 3.0 | 3.0 | 3.0 | 3.0 |
| Alkyl | — | <5 ppm | <5 ppm | <5 ppm | <5 ppm | <5 ppm |
| phenols | | | | | | |
| OPCs | — | 9 | 10 | 12 | 15 | 18 |

Test Results (in % inhibition compared to control)
Concentration of gingko extract in the test solution [% w/v]
Test in the presence of EDTA

| | | | | | | |
|---|---|---|---|---|---|---|
| 0.03 | 0 | 0 | 1 | 8 | 12 | 14 |
| 0.1 | 0 | 17 | 18 | 25 | 35 | 37 |

Test in the absence of EDTA

| | | | | | | |
|---|---|---|---|---|---|---|
| 0.003 | 0 | 8 | 10 | 37 | 45 | 49 |
| 0.01 | 0 | 36 | 40 | 76 | 79 | 81 |
| 0.03 | 0 | 68 | 70 | 79 | 80 | 82 |
| 0.1 | 0 | 73 | 75 | 83 | 85 | 86 |

*see explanation for Table I

### III.

### Superoxide and Hydrogen Peroxide Scavenging Activity

Two further ROS are superoxide (O₂.) and hydrogen peroxide (H₂O₂). Superoxide generated in vivo—for example during the oxidative burst of activated phagocytes, or in reactions involving cytochrom P450 oxidases—is largely converted enzmatically (SOD, superoxide dismutase) or by nonenzymatic dismutation to H₂O₂, which, being uncharged, is thought to cross cell membranes easily. For example, increased generation of O₂.⁻ and H₂O₂ in vascular tissues contribute to pro-inflammatory and other events related to vascular dysfunction and related disorders.

For the purpose of testing the ability of ginkgo extracts according to the present invention to scavenge O₂.⁻ and H₂O₂, these ROS can be generated using the xanthine oxidase/hypo-xanthin system and detected using chemiluminescense (luminol).

TABLE III
Superoxide and hydrogen peroxide scavenging activity of ginkgo
extracts with respect to variations in OPC content

| | | | | | |
|---|---|---|---|---|---|
| Control* | C1* | C2* | 1* | 2* | 3* |

Gingko extract composition (in % b.w.)

| | | | | | | |
|---|---|---|---|---|---|---|
| Flavone | — | 25.0 | 25.0 | 25.0 | 25.0 | 25.0 |
| glucosides | | | | | | |
| Ginkgolides | — | 3.0 | 3.0 | 3.0 | 3.0 | 3.0 |
| Bilobalides | — | 3.0 | 3.0 | 3.0 | 3.0 | 3.0 |
| Alkyl | — | <5 ppm | <5 ppm | <5 ppm | <5 ppm | <5 ppm |
| phenols | | | | | | |
| OPCs | — | 9 | 10 | 12 | 15 | 18 |

Test Results (in % inhibition compared to control)

| | | | | | | |
|---|---|---|---|---|---|---|
| 0.0001 | 0 | 36 | 38 | 50 | 65 | 67 |
| 0.001 | 0 | 72 | 75 | 83 | 86 | 89 |
| 0.01 | 0 | 100 | 100 | 100 | 100 | 100 |

*see explanation for Table I

### IV.

### Singlet Oxygen Quenching Activity

Singlet oxygen (¹O₂) is an electronically-excited form of molecular oxygen that may be generated in vivo either photochemically, i.e., upon exposure to light, or metabolically, for example by activated neutrophils, in the course of lipid peroxidation, and in enzymatic reactions related to anti-inflammatory mediators (prostaglandin) and detoxification (cytochrom P450 oxygenases). For the purpose of assessing the singlet oxygen-quenching activity of gingko extracts as described in this invention, the involvement of singlet oxygen in light-induced damage to the skin has been used. Light-induced damage to the skin—such as photoageing, also known as premature ageing of the skin—via singlet oxygen is mediated by both induction of enzymes involved in degradation of the dermal extracellular matrix, and by direct reactions with collagen, one of the skin's extracellular matrix proteins. Reactions include formation of aberrant crosslinks, thus disturbing the skin matrix integrity. For assessing the ¹O₂ induced collagen damage, ¹O₂ was generated in vitro via UVA irradiation using riboflavin as photosensitizer, and collagen damage was measured by the increase in viscosity of an aqueous solution of collagen and glucose. The results are given in Table IV.

TABLE IV
Singlet oxygen quenching activity of ginkgo extracts with respect to
variation in OPC content

| | | | | | |
|---|---|---|---|---|---|
| Control* | C1* | C2* | 1* | 2* | 3* |

Gingko extract composition (in % b.w.)

| | | | | | | |
|---|---|---|---|---|---|---|
| Flavone | — | 25.0 | 25.0 | 25.0 | 25.0 | 25.0 |
| glucosides | | | | | | |
| Ginkgolides | — | 3.0 | 3.0 | 3.0 | 3.0 | 3.0 |
| Bilobalides | — | 3.0 | 3.0 | 3.0 | 3.0 | 3.0 |
| Alkyl | — | <5 ppm | <5 ppm | <5 ppm | <5 ppm | <5 ppm |
| phenols | | | | | | |
| OPCs | — | 9 | 10 | 12 | 15 | 18 |

Test Results (in % inhibition compared to control)
Concentration of gingko extract in the test solution [% w/v]

| | | | | | | |
|---|---|---|---|---|---|---|
| 0.005 | 0 | 41 | 43 | 50 | 70 | 72 |
| 0.010 | 0 | 54 | 56 | 75 | 78 | 79 |
| 0.015 | 0 | 61 | 65 | 83 | 89 | 91 |

*see explanation for Table I

The results of the various tests show that gingko extracts with increased OPC content according to the present invention act as antioxidants against a variety of relevant ROS which are generated by the human body and via exogenous sources, and which contribute to oxidative stress-induced damage to important biomolecules and biosystems relevant to human health It should be well noted that the increase of advantageous properties does not simply follow a proportionality, but one can observe that there is a critical OPC concentration beginning at about 11 to 12% by weight.

The antioxidant effects of the gingko extracts with increased OPC content are displayed towards radicals in general, as demonstrated in the DPPH assay. Further, they involve scavenging of the hydroxyl radical (HO.), considered to be the most reactive of all ROS, which is generated in many pathways of human metabolism and is also thought to be the actual active principal mediating damage by superoxide (O₂.⁻) and hydrogen peroxide (H₂O₂). Also, the gingko extracts with increased OPC content were shown to possess metal chelating properties, thus being able to prevent generation of ROS catalysed by transition metal ions. In addition, the test results demonstrate superoxide and hydrogen peroxide scavenging and singlet oxygen (¹O₂) quenching properties of the extracts, i.e., their antioxidant activity against further ROS responsible for many aspects of free radical damage to the human body's cells and tissues. The results of the various tests clearly demonstrate that the new extracts according to the invention with their improved antioxidant activity are more suitable for use in oral preparations intended to control signs of ageing, environmental stress, inflammation, and other health conditions, specifically those related to eye health, an known extracts having a reduced OPC content.

### Example B1

### Encapsulation of the New Ginkgo Extract

In a 500 ml three-necked flask equipped with a stirrer and reflux condenser, 3 g of agar agar were dissolved in 200 ml boiling water. First a homogeneous dispersion of 10 g of glycerol in an additional 100 g water and then a preparation of 25 g of chitosan (Hydagen® DCMF, 1% by weight in glycolic acid [from Cognis Deutschland GmbH & Co. KG, Düsseldorf/FRG], 10 g of a spray-dried extract of Ginkgo biloba according to Example A1, 0.5 g of Phenonip® preservative mixture (containing phenoxyethanol and parabens) and 0.5 g of polysorbate 20 (Tween® 20, from ICI) in an additional 100 g of water were added to the mixture over a period of about 30 minutes with vigorous stirring. The matrix obtained was filtered, heated to 50° C. and dispersed with vigorous stirring in 2.5 times its volume of paraffin oil cooled beforehand to 15° C. The dispersion was then washed with an aqueous solution containing 1% by weight of sodium lauryl sulfate and 0.5% by weight of sodium alginate and then washed repeatedly with a 0.5% by weight aqueous Phenonip® preservative solution, the oil phase being removed in the process. An aqueous preparation containing 8% by weight microcapsules with a mean diameter of 1 mm was obtained after sieving.

### Example B2

### Encapsulation of the New Ginkgo Extract

In a 500 ml three-necked flask equipped with a stirrer and reflux condenser, 1 g of agar agar was dissolved in 33 g of water and heated to 100° C. Subsequently, 50 g of a 2% by weight aqueous solution of calcium alginate and 5 g of a 1% by weight aqueous solution of gellan gun (Kelgocel®, Degussa AG) was added. After vigorous sting 10 g of a spray-dried extract of Ginkgo biloba according to Example A1, 0.5 g of Phenonip® and 0.5 g of polysorbate 20 (Tween® 20, ICI) in an additional I 00 g water were added to the mixture over a period of about 30 minutes. The composition thus obtained was dropped into a bath consisting of a capric caprylic triglycerides (Myritol® 331, Cognis Deutschland GmbH & Co. KG). The resulting microcapsules of the agar/gellan gum/alginate-type were separated off and washed with an aqueous solution containing 1% by weight of polysorbate 20, in order to remove traces of the oil component. Subsequently, the soft capsules were introduced into a bath consisting of an aqueous 0.5% by weight solution of calcium chloride for cross-linking and hardening of the capsule walls. An aqueous preparation containing 8% by weight microcapsules with a mean diameter of 0.25 mm was obtained after sieving.

1. An exact from the leaves of Ginkgo biloba, comprising
(a) 20 to 30% by weight of flavone glycosides;
(b) 2.5 to 4.5% by weight in the aggregate of ginlkgolides A, B, C and J;
(c) 2.0 to 4.0% by weight of bilobalides;
(d) less than 10 ppm alkyl phenol compounds; and
(e) more than 10% by weight of oligomeric proanthocyanidins.

2. An extract according to claim 1, comprising 11 to 20% by weight of oligomeric proanthocyanidins.

3. An extract according to claim 1, comprising 12 to 18% by weight of oligomeric proanthocyanidins.

4. An extract according to claim 1, comprising less t 50 ppm 4′-O-methyl-pyroxidines within the oligomeric proanthocyanidins.

5. An extract according to claim 1, comprising less than 100 ppm biflavones within the oligomeric proanthocyanidins.

6. An extract according to claim 1, comprising 5 to 10% by weight of tetpene lactones within the oligomeric proanthocyanidins.

7. An extract according to claim 1, additionally comprising up to 5% by weight of water.

8. A capsule comprising one or more extracts according to claim 1.

9. A process for making an extract according to claim 1, comprising the following steps:
(i) subjecting ginkgo leaves or dry ginkgo eats of Ginkgo biloba to extraction with an aqueous polar solvent in order to give a liquid intermediate LI-1;
(ii) separating intermediate LI-1 from the aqueous polar solvent and subjecting it to a liquid-liquid extraction with a non-polar C₄-C₁₀ hydrocarbon in order to obtain an aqueous liquid intermediate LI-2;
(iii) subjecting intermediate LI-2, after adjusting its pH to 2.5-6, to a liquid-liquid extraction with a polar C₂-C₆ aliphatic alcohol in order to obtain an aqueous liquid intermediate LI-3, rich in oligomeric proanthocyanidins, and organic liquid intermediate LI4, rich in glycosides;
(iv) concentrating intermediate LI-4, diluting it with water and mixing it with a non-polar C₄-C₁₀ hydrocarbon in order to obtain an organic liquid intermediate LI-5 and an aqueous liquid intermediate LI-6;
(v) drying intermediate LI-6 to give a solid intermediate SI-1;
(vi) separating intermediate LI-3 from the polar C₂-C₆ aliphatic alcohol, diluting it with water, adjusting its pH to a value of 6 to 8, and cooling it to a temperature of, at most, 10° C. for a period sufficient to precipitate the oligomeric proanthocyanidins from intermediate LI-3;
(vii) filtering off the precipitated oligomeric proanthocyanidins, then washing and drying the oligomeric proanthocyanidins in order to obtain a solid intermediate SI-2; and
(viii) adding intermediate SI-2 to the intermediate SI-1 in an amount such that a final product of intermediates SI-1 and SI-2 contains more than 10% by weight of oligomeric proanthocyanidins.

10. A process according to claim 9, wherein the ginkgo leaves comprise flavone glycosides, ginkgolides and bilobalides in an amount of at least 10% by weight.

11. A process according to claim 9, wherein the dry ginkgo extracts comprise flavone glycosides, ginkgolides and bilobalides in an amount of 5 to 20% by weight.

12. A process according to claim 9, wherein the aqueous polar solvent of step (i) is acetone or ethanol.

13. A process according to claim 9, wherein the non-polar C₄-C₁₀ hydrocarbon of step (ii) and of step (iv) is n-heptane.

14. A process according to claim 9, wherein the polar C₂-C₆ aliphatic alcohol of step (iii) is n-butanol.

15. A pharmaceutical composition comprising one or more extracts according to claim 1.

16. A functional food composition comprising one or more extracts according to claim 1.

17. A functional food composition according to claim 16, wherein the one or more extracts are present, in an amount from 10 to 1,000 mg, based on the weight of the functional food composition.

18. A method of delivering an extract according to claim 1, comprising oral administration.

19. A method of improving retinal microcirculation by administering a pharmaceutical composition comprising one or more extracts according to claim 1.
